(19) United States
(12) Patent Application Publication
(10) Pub. No.: US 2023/0414463 A1
(43) Pub. Date: Dec. 28, 2023

(54) A THICKENED SILVER DIAMINE FLUORIDE COMPOSITION

(71) Applicant: ADVANTAGE SILVER DENTAL ARREST, LLC, Salem, OR (US)

(72) Inventor: Peter M. Milgrom, Seattle, WA (US)

(73) Assignee: ADVANTAGE SILVER DENTAL ARREST, LLC, Salem, OR (US)

(21) Appl. No.: 18/247,371

(22) PCT Filed: Oct. 8, 2021
(86) PCT No.: PCT/US2021/054175

§ 371 Date: Mar. 30, 2023

Related U.S. Application Data
(60) Provisional application No. 63/091,964, filed on Oct. 15, 2020.

(51) Int. Cl. A61K 8/21; A61Q 11/00; A61K 8/04

(52) U.S. Cl. CPC A61K 8/21; A61K 2800/43; A61K 8/042; A61Q 11/00

(57) ABSTRACT

A composition for oral application comprising silver diamine fluoride that is thixotropic and has a viscosity of 0.02 to 500 Pa·s as measured by a TA AR-G2 rheometer with a 40 mm cone and plate, carried out at a temperature of about 23 #C, and a shear rate of from 0.1/second to 100/second. The thixotropic properties allow for improved application by a dentist.

### CROSS REFERENCE TO RELATED APPLICATIONS

The present application claims priority to U.S. Provisional Patent Application No. 63/091,964, filed Oct. 15, 2020, the entire contents of which are fully incorporated herein by reference.

### TECHNICAL FIELD

The present disclosure relates generally to oral treatment. More specifically, the present disclosure relates to a composition for coating a decayed portion of tooth comprising a silver diamine fluoride comprising, optionally, a coloring agent, and a thickening agent, such as a cellulose derived component that provides increased viscosity and aids a dentist to control the application of the composition. In addition, the present disclosure relates to a composition for prevention of lesions in high-risk teeth. For example, patients undergoing head and neck radiation for mouth cancer have teeth that are at a high risk of developing carious lesions. A second example of high-risk teeth are those with exposed root surfaces that are serving as abutments for fixed or removable partial dentures. A third example an at risk tooth is where the mesial proximal surface of a permanent first molar in a child abuts a dental caries lesion in the adjacent primary second molar.

### BACKGROUND OF THE INVENTION

Dental caries is a common chronic disease in both children and adults. Products of metabolism by bacteria populating the tooth surface induce development and progression of cavities. There are a couple of ways of treating cavities, for example removal of carious tissues with drills, and more recently, the use of lasers to remove affected tissue. Both methods are invasive and may be extremely painful and unpleasant for a patient.

More recently, the application of silver diamine fluoride (SDF) has been employed as an alternative. However, silver diamine fluoride contains ammonia and has a low viscosity, and thus is a liquid at room temperature. Therefore, the application of this material must be carried out carefully as it is difficult to control the intended location of the liquid on the tooth at the time of application. If the liquid runs off the intended location on the tooth, it may come in contact with gums, other mucosal surfaces, tongue, lips, and face. Furthermore, the silver diamine fluoride may result in the formation of silver oxide which may result in a pigmentation of the applied area. This may result in a range of brown to dark brown discoloration which is undesirable on certain affected areas, including the skin.

EP 2 794 028 relates to an oral care composition comprising a fluoride ion source, a poly(propylene oxide)/poly(ethylene oxide) copolymer, and additional components such as carboxymethyl cellulose.

U.S. 2018/0280431 relates to a composition for preventing the occurrence of tooth lesions caused by dental caries comprising metal particles, a polysaccharide chitosan, a fluoride salt, an organic acid, a liquid dispersing agent, and a flavoring agent.

Accordingly, what is needed is a composition that has an improved viscosity which allows for a dentist to control the application of the formulation to the intended area without inadvertent extension to other oral structures or spills on the face or equipment.

Additional features of the present disclosure will become apparent to those skilled in the art upon consideration of the following detailed description of the presently perceived best mode of carrying out the disclosure.

### SUMMARY AND TERMS

The inventors have surprisingly found that adding a thickening agent to the silver diamine fluoride can meet these needs with no evidence of unintended adverse effects, while maintaining the desirable properties of the SDF.

In a first aspect, the disclosure relates to a composition for oral application comprising silver diamine fluoride which is thixotropic and has a viscosity of 0.02 to 500 Pa s, or from 0.1 to 500 Pa·s, or from 0.1 to 450 Pa·s as measured by a TA AR-G2 rheometer with a 40 mm cone and plate, and carried out at a temperature of about 23° C., and a shear rate of from 0.1/second to 100/second.

In a second aspect, the disclosure relates to a composition for oral application comprising silver diamine fluoride and a thickening agent, wherein the thickening agent is in an amount of 0.1 to 15 wt. %, or 0.25 to 10 wt. % or 0.5 to 5 wt. %, based on a total weight of the composition.

In each of the foregoing embodiments, the silver diamine fluoride may be combined with the composition in the form of an aqueous solution having a concentration of from 10 to 40 wt. %, based on the total weight of the composition, preferably, the silver diamine fluoride is present in a concentration of about 38% wt. %, based on the total weight of the composition. The silver diamine fluoride may provide a silver concentration of about 20 wt. % (200,000 ppm) to 30 wt. % (300,000 ppm), or about 25 wt. % (250,000 ppm), an ammonia concentration of 5 wt. % (50,000 ppm) to 10 wt. % (100,000 ppm), or about 8 wt. % (80,000 ppm), and a fluoride concentration of 1 wt. % (10,000 ppm) to 10 wt. % (100,000 ppm), or about 5 wt. % (50,000 ppm), to the composition. The aqueous silver diamine fluoride solution provides about 50,000 ppm of fluoride to the total weight of the composition. The silver diamine fluoride solution is available as Advantage Arrest Silver Diamine Fluoride 38 wt. %, Riva Star Silver Diamine Fluoride 38 wt. % with Potassium Iodide (saturated solution), Fluoroplat SDF (South America), and eSDF (India). In another embodiment, the composition comprises silver diamine fluoride in an amount of from 10 wt. % to 40 wt. %, or about 38 wt. %, a thickening agent in an amount of from 0.1 wt. % to 15 wt. %, or from about 0.5 wt. % to about 5 wt. %, other additives, such as a coloring agent, stabilizing agent, mixing agent, and flavoring agent in an amount of from about 0 wt. % to about 10 wt. %, and a balance of water, to arrive at a total weight percent of 100, all based on the total weight of the composition. Preferably, the composition comprises silver diamine fluoride in an amount of about 38 wt. %, a thickening agent in an amount of about 1.5 wt. %, and water in an amount of about 60.5 wt. %, based on the total weight of the composition.

In each of the foregoing embodiments, the silver diamine fluoride composition may include a thickening agent.

In each of the foregoing embodiments, the silver diamine fluoride composition may have the thickening agent in an amount of 0.1 to 15 wt. %, or 0.25 to 10 wt. %, or 0.5 to 5 wt. %, based on the total weight of the composition.

In each of the foregoing embodiments, the thickening agent may be a cellulose derivative (“cellulose gum”) such as carboxymethyl cellulose (CMC), methyl cellulose, hydroxyethyl cellulose, hydroxypropyl methyl cellulose, microcrystalline cellulose, and mixtures thereof, polyvinyl pyrrolidone; xanthan; an admixture of glycerin and polyacrylate; carrageenans such as iota-carrageenan, kappa-carrageenan, kappa2-carrageenan, lambda-carrageenan, and mixtures thereof, guar gum; gum karaya; gum arabic; gum tragacanth; and mixtures thereof, hydrated silica and colloidal silica may be used as thickeners, or Silica thickeners.

In each of the foregoing embodiments, the thickening agent may be selected from carboxy methyl cellulose (CMC), microcrystalline cellulose, and an admixture of glycerin and polyacrylate, and more preferably, carboxy methyl cellulose.

In each of the foregoing embodiments, the silver diamine fluoride composition may be prepared by mixing silver diamine fluoride with the thickening agent, wherein the silver diamine fluoride is in an amount of about 38 wt. % and the thickening agent is carboxy methyl cellulose (CMC) in an amount of 0.1 to 5 wt. % based on the total weight of the composition.

In each of the foregoing embodiments, the silver diamine fluoride composition may further include a clathrate of glyceryl acrylate and glyceryl polyacrylate that encloses water molecules, or the clathrate is a cyclodextrin which includes α-cyclodextrin, β-cyclodextrin, and γ-cyclodextrin, and derivatives of cyclodextrin such as methylated cyclodextrins, ethylated cyclodextrins, hydroxypropyl cyclodextrins, and hydroxyethyl cyclodextrins, wherein the clathrate is present in an amount of from 0.1 to 15 wt. %, or 0.25 wt. % to 10 wt. %, or 0.5 to 5 wt. %, based on the total weight of the composition.

In each of the foregoing embodiments, the silver diamine fluoride composition may further include a coloring agent, wherein the coloring agent may be triarylmethane dye.

In each of the foregoing embodiments, the silver diamine fluoride composition may further include a flavoring agent.

In each of the foregoing embodiments, the silver diamine fluoride may be a thixotropic fluid that can undergo an isothermal gel-sol-gel transformation.

In each of the foregoing embodiments, the silver diamine fluoride may have a pH of no lower than 7.0, or a pH of from 7.0 to about 13.5, or from about 10.0 to about 10.5. The additives can themselves have varying pH while being able to withstand high pH of SDF which is 10-10.5.

In each of the foregoing embodiments, the silver diamine fluoride composition may have a viscosity of greater than 75 Pa·s, or greater than 100 Pa·s, or greater than 125 Pa·s at a shear rate of 0.1/sec and less than 15 Pa·s, or less than 10 Pa·s, or less than 8 Pa·s at a shear rate of 100/sec.

In a third aspect, the present disclosure relates to a method of treating teeth, comprising applying an effective amount of the composition of any one of the foregoing embodiments to a decayed portion of tooth.

In a fourth aspect, the present disclosure relates to a method of preventing caries lesions in high-risk teeth, comprising applying an effective amount of the composition of any one of the foregoing embodiments to a high-risk tooth. For example, patients undergoing head and neck radiation for mouth cancer are at a high risk of developing caries lesions. A second example of high-risk teeth are those with exposed root surfaces that are serving as abutments for fixed or removable partial dentures. A third example an at risk tooth is where the mesial proximal surface of a permanent first molar in a child abuts a dental caries lesion in the adjacent primary second molar.

The following definitions of terms are provided in order to clarify the meanings of certain terms as used herein.

The term “dental caries”, commonly referred to as tooth decay, is a disease in which damage to the tooth structure occurs. The damaged tooth structure is called a cavity or dental caries lesion that is caused by acid released from a bacteria colonizing the tooth surface. The tooth includes, in part, the enamel, the dentin, the cementum, and the pulp. The enamel comprises the outer surface of the crown of the tooth, and the dentin is the layer just below the enamel. The cementum covers the root surface. The pulp is the central part of the tooth, which includes soft connective tissue, blood vessels and nerves. Dental caries, as used herein, refers to destruction or decay of the enamel, dentin, cementum and/or pulp or any combination thereof. Carious lesions refer to injury to the tooth structure that is caused by dental caries.

The presence, absence, or state of caries lesions can be determined by a health professional or lay person using methods that are known in the art. For example, early dental caries is determined by a visual identification of “white spot” lesions. Caries lesions are also determined by visual and tactile exam identifying discolored or decalcified pits and fissures. Frank cavitation is identified as a clear break in the enamel. The presence of white spots discolored or decalcified pits and fissures, or frank cavitation indicates the presence of dental caries. Inspection of visible tooth areas can be performed with a dental mirror and explorer. Caries can be identified by its texture and architecture. Healthy enamel and dentin are denser to probing with a dental instrument, i.e., dental explorer, as compared to enamel and dentin that are infected with dental caries. Additionally, caries lesions can be diagnosed with use of X-rays, especially in areas that are not easily visible. Other technologies such as fiber optic illumination, lasers, and dyes can also be used to identify the presence or absence of dental caries lesions.

The present invention further includes treating dental caries. “Treating” dental caries refers to the prevention or cessation or reduction of progression of caries lesions. Treating dental caries includes preventing the carious lesion from beginning or getting worse. For example, the carious lesion is treated when the lesion does not get larger in size and/or does not further affect additional tooth structure (e.g., penetrate from the enamel to the dentin).

As used herein, the term “high-risk teeth” refers to teeth that are at a higher risk of developing caries lesions than the average person's tooth. For example, patients undergoing head and neck radiation for mouth cancer have teeth that are at a high risk of developing caries lesions. A second example of high-risk teeth are those with exposed root surfaces that are serving as abutments for fixed or removable partial dentures. A third example an at risk tooth is where the mesial proximal surface of a permanent first molar in a child abuts a dental caries lesion in the adjacent primary second molar.

As used herein, the term “effective amount” means an amount of a composition comprising an antimicrobial material, for example, SDF, that is effective to prevent or arrest formation of dental caries. Such a composition may also include one or more additional active ingredients, including without limitation one or more inactive ingredients, as discussed below.

### BRIEF DESCRIPTION OF THE DRAWINGS

FIG. 1 is a graph of relative drop volume over time for the experimental 38% SDF gel formulation (A) and the positive control non-viscous conventional 38% liquid (B). The value of the relative drop volume is related to the respective initial volume of the drop (which is set as 100%).

FIG. 2 is a representative surface imaging by SEM at a magnification of 2,500× in backscattered electron mode (material contrast) of treated dentin samples are shown in the upper row. The chemical element mapping is performed using the silver signal. FIGS. 2a and 2b show the dentin samples treated with the experimental 38% SDF gel formulation (A), FIGS. 2c and 2d show the dentin samples treated with placebo (C) and FIGS. 2e and 2f show the dentin samples treated with positive control non-viscous conventional 38% liquid (B). The lower row shows element maps of the corresponding SEM-micrographs of upper row. Scale bar=25 μm.

FIG. 3 is a representative surface imaging by SEM at a magnification of 25,000× in backscattered electron mode (material contrast) of treated dentin samples, scale bar 2.5 μm. In FIG. 3a tubule occlusions as well as openings of tubules with a relatively small diameter are visible using non-viscous conventional 38% liquid (B). Open (seemingly unaffected) tubules are visible in the SEM micrograph of FIG. 3b using placebo (C). The SEM micrograph of FIG. 3c shows silver precipitates with clearly elevated structure on the dentin surface using experimental viscous 38% SDF gel (A).

FIG. 4 shows representative cross section images of fractured dentin samples by SEM in backscattered electron mode. Images of the upper row show an overview (scale bar corresponds to 100 μm), whereas images of the lower row depict a detailed view in higher magnification (scale bar corresponds to 20 m). FIGS. 4a and 4b show the dentin samples treated with experimental 38% SDF gel formulation (A), FIGS. 4c and 4d show the dentin samples treated with placebo (C) and FIGS. 4e and 4f show the dentin samples treated with non-viscous conventional 38% liquid (B). The sample's surface is indicated by arrows.

### DETAILED DESCRIPTION OF THE INVENTION

In a first aspect, disclosed is a composition for oral application comprising silver diamine fluoride and having a viscosity of 0.02 to 500 Pa·s. The oral application may further comprise a thickening agent. It was found that adding a thickening agent to the silver diamine fluoride improves the ability of a dentist or user to control the application of the oral composition.

Silver diamine fluoride is an effective agent against dental caries when applied every 12 months, with proven action in the prevention of coronal caries. Bacterial pathogens that colonize and produce acids as a by-product of their metabolism demineralize the tooth and result in the cavity. The bacterially produced acids further soften the inside of the cavity. The SDF is employed to kill the bacteria and arrest the progression of the acid destruction. The fluoride causes the formation of calcium fluoride which serves as a fluoride reservoir to promote remineralization of lesions and prevent them forming in other teeth in the mouth. In addition, the SDF reinforces and hardens the lesion, and this is theorized to be caused by the formation of silver complexes (sometimes called microwires) in the voids left in the structure by the acid destruction. The silver compounds may be found in the tubular structure of the dentin or inner part of the tooth, resulting in decreased thermal and mechanical hypersensitivity of the teeth during eating or drinking or dental treatment. The SDF compound is found for distribution in the form of aqueous solutions with concentrations ranging from 10 wt. % to 40 wt. %, but preferably is present in a concentration of about 40 wt. %. The terms “silver diamine fluoride” or “silver diammine fluoride” or “diammine silver fluoride”, “AgF(NH₃)₂” may be used interchangeably.

The thickening agent is present in an amount such that the composition for oral application may be thixotropic and have a viscosity of from 0.02 to 500 Pa·s, or from 0.1 to 500 Pa·s, or from 0.1 to 450 Pa·s, as measured by a TA AR-G2 rheometer with a 40 mm cone and plate, carried out at room temperature (about 23° C.), and a shear rate of 0.1/second to 100/second. In some embodiments, the silver diamine fluoride composition may have a viscosity of greater than 75 Pa·s, or greater than 100 Pa·s, or greater than 125 Pa·s at a shear rate of 0.1/sec and less than 15 Pa·s, or less than 10 Pa·s, or less than 8 Pa·s at a shear rate of 100/sec. Suitable examples of thickening agents may be selected from cellulose derivative (“cellulose gum”) such as carboxymethyl cellulose (CMC), methyl cellulose, hydroxyethyl cellulose, hydroxypropyl methyl cellulose, microcrystalline cellulose, and mixtures thereof; polyvinyl pyrrolidone; xanthan; an admixture of glycerin and polyacrylate; carrageenans such as iota-carrageenan, kappa-carrageenan, kappa2-carrageenan, lambda-carrageenan, and mixtures thereof, guar gum; gum karaya; gum arabic; gum tragacanth; and mixtures thereof, hydrated silica and colloidal silica may be used as thickeners, or Silica thickeners. Preferably, the silver diamine fluoride composition does not include poly(propylene oxide)/poly(ethylene oxide) copolymers or generally, poloxomers. Preferably, the thickening agent is a carboxymethyl cellulose. The thickening agent may be present in an amount of 0.1 to 15 wt. %, or 0.25 to 10 wt. %, or 0.5 to 5 wt. %, based on the total weight of the composition.

In some embodiments, the thickening agent may act as a thixotropic additive. Thixotropic additives are capable of integrating a compound that is capable of polymerizing or that has already partly or fully, polymerized, and has at least two groups available for hydrogen bonding, into a gel-like three-dimensional network. Thixotropic additive will impact a composition to have time-dependent shear thinning properties. Therefore, the oral composition may be thick or viscous under static conditions, however the composition may flow (become thinner, less viscous) over time when shaken, agitated, shear-stressed, or otherwise stressed. This property enables application of a thixotropic mixture as a semi-solid state to a body surface, which subsequently becomes substantially liquid and therefore more spreadable when applied to a surface, for example, on a tooth.

More specifically, when the composition of the present invention is sheared by squeezing the product bottle, the weak chemical bonds are broken, and a lyophobic solution is formed that can be applied to the decayed tooth. Once applied to the tooth, the particles collide, flocculation occurs, and the gel is reformed such that the dental provider can control the application.

The composition of the present invention may be thixotropic, and thus capable of undergoing an isothermal gel-sol-gel transformation. In one or more embodiments, when poured, it displays flow, but over time it reverts to being more viscous or gel like. In one or embodiments, when shear force is applied it displays flow, but overtime reverts to being more viscous or gel like. In one or more embodiments, a solid gel becomes flowable and later with time become solid or semi-solid. In one or more embodiments a semi-solid gel becomes flowable and later with time become solid or semi solid. In one or more embodiments, a liquid gel is flowable and later with time becomes solid or semi solid.

Thickening agents, which may also act as thixotropic additives may be selected from carboxymethyl cellulose, carrageenan, and silica. Thickening agents, such as xantham gum typically do not impart thixotropy properties when added to compositions.

The composition of the present invention may also include clathrates. Clathrates consist of a lattice that is capable of trapping or containing molecules. Preferably, the clathrates of the present invention comprise glyceryl acrylate and glyceryl polyacrylates, suitable for enclosing water molecules via hydrogen bonding and Van der Waals forces. Preferably, the clathrate of glyceryl acrylate and glyceryl polyacrylate contains about 50 wt. % of water. The clathrate may exhibit shear-thinning properties, similar to natural saliva. Another example of a clathrate suitable for the present invention is a cyclodextrin. Examples of suitable cyclodextrins include α-cyclodextrin, β-cyclodextrin, and γ-cyclodextrin, and derivatives of cyclodextrin such as methylated cyclodextrins, ethylated cyclodextrins, hydroxypropyl cyclodextrins, and hydroxyethyl cyclodextrins. The clathrates of the present invention may be present in an amount of from 0.1 to 15 wt. %, or 0.25 to 10 wt. %, or 0.5 to 5 wt. %, or 0.1 to 5 wt. %, based on the total weight of the composition.

In some embodiments, the composition of the present invention may be prepared by mixing 38 wt. % silver diamine fluoride (aqueous) with the thickening agent, wherein the thickening agent is carboxy methyl cellulose (CMC) in an amount of from 0.1 to 15 wt. %, or 0.25 to 10 wt. %, or 0.5 to 5 wt. %, or 0.1 to 5 wt. %, based on the total weight of the composition. Enough water can be removed from the composition to compensate for the added CMC so that the final gel composition has 38 wt. % silver diamine fluoride.

In an exemplified embodiment, the composition or “gel formula” is formed by mixing an aqueous silver diamine fluoride 38 wt. % solution with carboxymethyl cellulose (CMC) as the thickening agent, wherein the final gel composition has 1.5 wt. % CMC. In another exemplified embodiment, the gel formula is formed by mixing silver diamine fluoride 38 wt. % with carboxymethyl cellulose as the thickening agent, and a clathrate of glyceryl acrylate and glyceryl polyacrylate. Preferably, the clathrate is added during a cooling phase using low-shear mixing. Enough water can be removed from the composition to compensate for all additives so that the final gel composition has 38 wt. % silver diamine fluoride.

The compositions of the present invention have a pH of no lower than 7.0, or a pH of from about 7.0 to about 13.5, or a pH of from 7.0 to 10.5, or a pH of from 10-10.5. The additives can themselves have varying pH while being able to withstand high pH of SDF which is 10-10.5.

In some embodiments, the present invention may include a color agent to improve the appearance. A side effect of silver diamine fluoride may be the formation of silver oxide which may result in a pigmentation of the applied area. A coloring agent such as titanium dioxide, triarylmethane dye (FD&C no. 1), FD&C no. 2, FD&C no. 3, FD&C no. 4, FD&C no. 5 may be included to mask the brown color resulting from the silver oxide. Preferably, the coloring agent is triarylmethane dye. As the triarylmethane dye breaks down slowly over time, the brilliant blue dye may color shift to purple or red, indicating that it is expired.

The compositions of the present invention may also include a flavoring agent. Suitable flavoring agents may be selected from, but are not limited to, essential oils, as well as various flavoring aldehydes, esters, alcohols, and similar materials. Examples of essential oils include oils of spearmint, peppermint, wintergreen, sassafras, clove, sage, eucalyptus, marjoram, cinnamon, lemon, lime grapefruit, and orange. The flavoring agent may be included in the oral composition at concentrations of approximately 0.1 to about 5.0 wt. % by weight, or from about 0.5 to about 1.5 wt. %, based on the total weight of the oral composition.

The compositions of the present invention may also include other additives, such as mixing and stabilizing agents.

In another aspect, the present invention relates to a method of treating teeth, comprising applying the oral compositions disclosed herein, to a decayed portion of tooth.

Rheology of Gel Compositions and Gel Properties Rigidity and viscosity are two separate rheological parameters used to characterize the mechanical properties of the present invention, which may be a gel. Preferably, the compositions of the present invention possess the following properties:
1. Uniformity: the composition should be formulated so that it is and can remain uniform without separation or precipitation over time.
2. Flowability: The composition, when placed in a tube or container and expelled under shear force should be flowable.
3. Stability/Breakability: The fine balance between stability and breakability of the gel coming out of the tube or container is very delicate: on the one hand, the gel should preferably not be very runny upon release from the tube or container and not lose its thixotropy property as a result of exposure to a tooth; and on the other hand, it should be “breakable”, i.e., it should spread easily, break down and absorb onto the surface upon application of mild shear force.

### EXAMPLES

### Viscosity

Gels can be analyzed on a RA AR-G2 rheometer using a 40 mm cone and plate rheometer, carried out at shear rates from 0.1/second to 100/second and at room temperature (about 23° C.). Gels A-D are expected to demonstrate significant thixotropic behavior as follows.

| Sample | Gel A | | Gel B | | Gel C | | Gel D | |
|---|---|---|---|---|---|---|---|---|
| Shear rate | 0.1/s | 100/s | 0.1/s | 100/s | 0.1/s | 100/s | 0.1/s | 100/s |
| Viscosity (cps) | 325,000 | 4,000 | 135,000 | 3,800 | 208,000 | 2,300 | 420,000 | 3,000 |

### Dentin Tubule Occlusion—an In Vitro Investigation

As mentioned above, the conventional 38% silver diamine fluoride (SDF) is effective in treatment of dentin hypersensitivity and caries lesions. However, the conventional formulation is a non-viscous solution that does not easily allow clinicians to control the application area. A 38% SDF experiment gel according to the present invention was compared in vitro to the conventional SDF formulation for its ability to penetrate and occlude dentinal tubules.

### Materials and Methods:

Human root surface dentin specimens were treated with gelled or standard 38% SDF or negative control. Penetration behavior was established using Drop Shape Analysis on naïve surfaces. In an artificial dental caries model, precipitates at the surface and within tubules were analyzed using SEM and EDX after treatment. Specifically, twenty-eight 4 mm×4 mm human dentin specimens were prepared from human tooth roots to mimic exposed dentin after gingival recession. The specimens were polished using 1200-grit paper until most of the dentin surface was flattened. The specimens were then serially polished using 4000-grit paper (Struers Inc.) followed by a 1-μm diamond polishing suspension (DP Suspension P, Struers).

Ten untreated specimens were then set aside for the penetration study. To open the dentinal tubules of the remaining 18 tooth specimens, all were immersed in 17% EDTA solution (pH 7.4; Fisher Scientific) for five minutes. They were then randomized and allocated 1:1:1 to the test groups before being treated with either one drop of an experimental, viscous (˜30 cP at room temperature) 38% SDF solution (labeled A), the positive control that is the conventional non-viscous 38% SDF solution (labeled B), or an aqueous placebo solution that contained neither fluoride nor silver (labeled C). The SDF solutions were allowed to remain on the exposed surfaces for one minute and then rinsed with running deionized water for 5 s. The test products and placebo were freshly prepared and certified by an FDA-regulated laboratory independent of the investigators, and the investigators were blind to the contents. The specimens were then immersed in artificial saliva (2.2 g/L gastric mucin, 0.381 g/L NaCl, 0.213 g/L CaCl₂·H₂O, 0.738 g/L K H₂PO₄, 1.114 g/L KCl, pH 7.0; Fisher Scientific) for 2 h, rinsed again with running deionized water for 5 s and stored at approximately 100% relative humidity.

### Penetration Behavior:

Contact angle measurements were performed using a Drop Shape Analyzer system (DSA 100, Top View Analyzer TVA 100, Krüss, equipped with software KRÜSS ADVANCE 1.10.0.34701). The application of drops was performed in a water vapor saturated environment at room temperature. Specifically, 0.3 μL drops of each formulation were placed on the sound dentin surface with a micropipette. The kinetics of drop shape change was recorded at intervals of 20 s for 400 s. Since a spreading effect was not observable, a reduction of the contact angle equal to volume reduction was measured. This is expressed as the percent shape/volume decrease. The respective data were averaged and presented with standard deviation for the corresponding time points. Five dentin samples which were prepared as described above were used for testing each formulation.

### Dentin Structure, Deposit Localization, and Deposit Identification:

Scanning electron microscopy (SEM) was used to characterize the morphology of surfaces and deposits with a SEM-FIB-EDX, Quanta3D FEG Dual-Beam (FEI Company). An acceleration voltage of 10 kV was applied. Energy-dispersive X-ray spectroscopy (EDX) was used to determine the nature of deposits used the Oxford Xplore EDX-Detector (Oxford Instruments) both on surfaces and cross-sectionally. The surface analysis was designed to evaluate deposits formed on the dentin surface region near the dentinal tubules and on the inter-tubular regions. Cross-sectionally fractured samples were used to detect silver deposits within the dentin tubules and to estimate their depth of penetration. The specimens for cross-section analysis were pre-sawed on the backside (opposite site of treatment) to ensure crack initiation. All specimens were coated with an ultra-thin platinum coating before SEM measurements.

For the surface analysis, nine SEM images were taken randomly at the center of the treated dentin surface and combined using imagery software (cellF, Olympus Soft Imaging Solutions), and then transformed into a binary image (binarized) so that white and black surface elements (white: precipitated silver particles and black: open tubules) could be differentially counted.

### Results

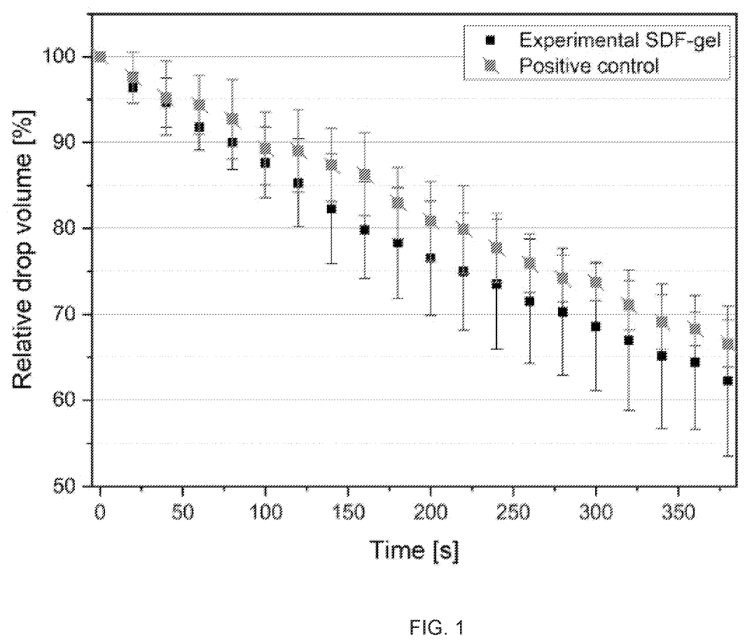

FIG. 1 describes the penetration of the two SDF formulations over time. The drop volume decreased immediately after placing it. The penetration of the two 38% SDF formulations was similar despite the fact that the inventive formulation (A) (referred to in FIG. 1 as “Experimental SDF-gel”) is more viscous than the non-viscous “Positive control” conventional 38% SDF liquid (B).

### Surface Precipitation and Tubule Occlusion:

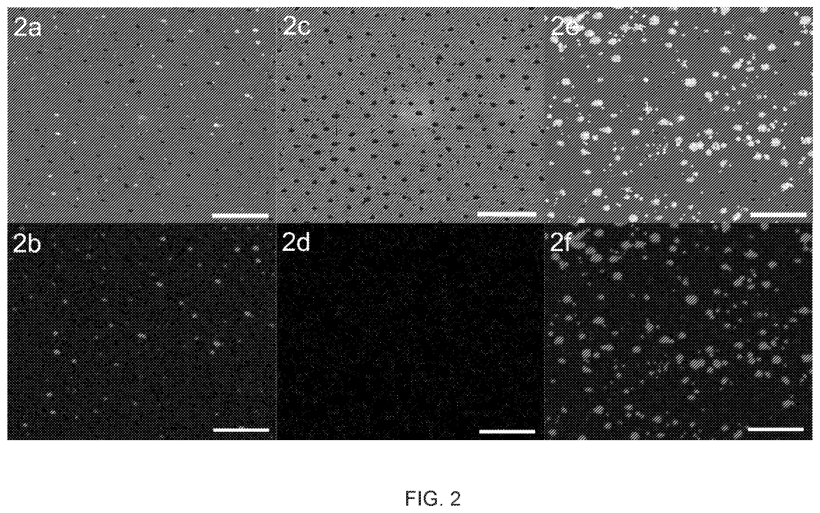

After application, surface precipitation and tubule occlusion were observed consistently for the positive control conventional 38% SDF non-viscous liquid (B) and the experimental viscous 38% SDF gel (A) but not for the placebo (C). Representative micrographs are shown in FIG. 2 (a,c,e). In FIGS. 2a and 2c, the bright structures represent the deposited silver on the dentin surface. The dark structures suggest open tubules. The surface of the placebo-treated dentin is characterized by open dentin tubules. Besides occluded tubules, open tubules are also visible on the dentin surface for groups A and B. EDX-mapping was done on the same samples (FIG. 2 b, d, f). Both SEM and EDX images give the impression of slightly higher silver precipitation on the dentin surface for test group A. This was confirmed quantitatively by software-based image analyses: the average percentage of dentin tubules occluded by silver precipitates was 31.4%±7.9% for group B and 62.6%±24.8% for group A; however, the difference was not statistically significant (p=0.11).

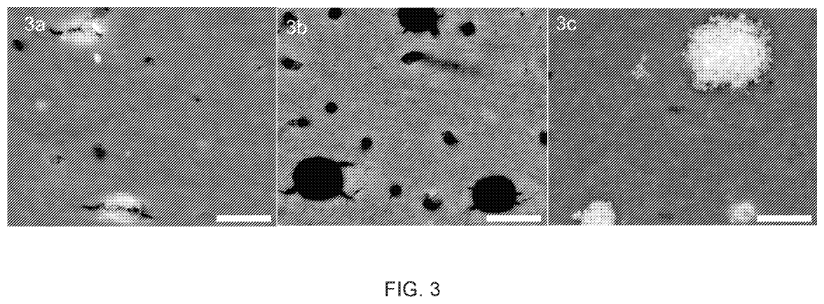

The morphologies of the silver precipitates on the dentin surface differed slightly for the two 38% SDF solutions. Associated with the slightly higher amount of superficially deposited material, the silver precipitates show more elevated structure on the dentin surface after treatment with experimental viscous 38% SDF gel (A) (see FIG. 3). In FIG. 3a tubule occlusions as well as openings of tubules with a relatively small diameter are visible using non-viscous conventional 38% liquid (B). Open (seemingly unaffected) tubules are visible in the SEM micrograph of FIG. 3b using placebo (C). The SEM micrograph of FIG. 3c shows silver precipitates with clearly elevated structure on the dentin surface using experimental viscous 38% SDF gel (A).

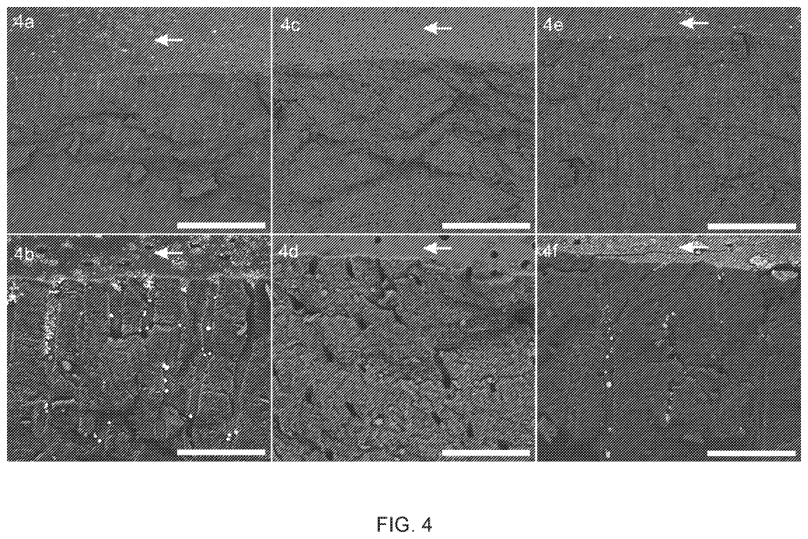

As evident in cross sectional images, silver precipitates in the form of particles can be observed at the surface and within dental tubules for samples A and B in FIG. 4.

Silver particles were not homogeneously distributed within a specimen, as was true for all samples in groups A and B. There were regions within the tubules with higher numbers of particles that appear to form chains of particles, and regions of lower numbers where isolated silver particles were seen. These particulate precipitates were found in almost every tubule, even when there was seemingly no tubule occlusion on the surface. Particle sizes ranged from nearly the diameter of a tubule to far below 1 m. The number of particles decreased with depth. The depth of penetrated silver particles was measured as up to 500 m (not depicted by the SEM images). There was no qualitative difference between them in groups A and B. For all C samples, no particulate precipitations were observed within the dentin tubules which is consistent with the fact that placebo (C) contained neither fluoride nor silver.

### Discussion

In this in vitro study the authors provided further evidence for the ability of 38% SDF to occlude dentin tubules and thereby prevent dentin hypersensitivity and arrest dental caries lesions. Furthermore, the behavior of the experimental viscous 38% SDF gel formulation (A) appeared to be indistinguishable from the conventional non-viscous 38% SDF liquid product (B).

Despite the differences in viscosity between the test gel product (A) and the conventional formulation (B), there were no significant differences in the penetration behavior. This is unexpected since the higher viscosity formulation (A) was expected to have more difficulty entering the small orifices of the dentin tubules. The data scatter can be attributed to the apparent variability of the dentin samples. The measured volume reduction principally is a result of the penetration of the formulation into the dentin. In the period critical to clinical application the standard SDF formulation and prototype showed comparable penetration kinetics.

Tubule occlusion was observed with both the experimental, more viscous form of 38% SDF formulation (A) and the conventional 38% SDF liquid product (B); considerable, although not statistically significant differences were observed between both SDF formulation in surface precipitation. Particulate deposits in the lumens and within the length of the tubules may form a mechanical blockade that could reduce fluid movement inside dentin tubules and thereby relieve dentin hypersensitivity. Although the surface analysis suggests that there are a number of seemingly non-occluded tubules, cross-sectional analysis reveals that silver penetrates in almost every tubule and precipitates within them.

Regarding penetration behavior and the resulting particle precipitation, i.e., number, size and distribution of the precipitates within the tubules, both 38% SDF formulations (A) and (B) exhibited comparable penetrations. From a morphological point of view, the particles in general had spherical shape; however, larger particles were more angular, suggesting crystallization processes occur, similar to what has been reported for insensitive dentin.

Penetration depths of around 500 μm were observed for both test products.

In summary, penetration depths up to 500 μm were observed for both SDF formulations (A) and (B) despite the expected barrier to penetration due to the higher viscosity of inventive formulation (A). Both formulations occluded dentinal tubules similarly. Precipitates on the dentin surface and within dentinal tubules were found for both SDF formulations, with a slight tendency for the experimental gel SDF product to be more abundant than the conventional one.

### SDF for Prevention of Caries in High-Risk Teeth

As mentioned above, silver diamine fluoride (SDF) can be used to prevent caries lesions in high-risk teeth. While SDF is almost completely absorbed into the tooth when applied to carious lesions, not as high of a proportion will be absorbed when applying to sound surfaces for prevention. Thus, when using conventional non-viscous SDF, there may be an increase in the amount that will interact with the soft tissues and possibly be absorbed into the systemic circulation due to spillage. On the other hand, the SDF composition of the present invention is non-viscous and is more easily applied to the high-risk surface without contamination of the unintended surfaces.

A dosage can be about 200-400 ppm SDF per 10 kg of body weight per visit is recommended for infants and toddlers. Teeth tend to erupt very early in the populations who experience the highest prevalence of severe early childhoods caries (e.g. Native American children). The visits can be limited to once or twice a year.

While this disclosure has been described as having an exemplary design, the present disclosure may be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains.

Other embodiments of the present disclosure will be apparent to those skilled in the art from consideration of the specification and practice of the embodiments disclosed herein. As used throughout the specification and claims, “a” and/or “an” may refer to one or more than one. Unless otherwise indicated, all numbers expressing quantities of ingredients, properties such as molecular weight, percent, ratio, reaction conditions, and so forth used in the specification and claims are to be understood as being modified in all instances by the term “about,” whether or not the term “about” is present. Accordingly, unless indicated to the contrary, the numerical parameters set forth in the specification and claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. It is intended that the specification and examples be considered as exemplary only, with a true scope and spirit of the disclosure being indicated by the following claims. The foregoing embodiments are susceptible to considerable variation in practice. Accordingly, the embodiments are not intended to be limited to the specific exemplifications set forth hereinabove. Rather, the foregoing embodiments are within the spirit and scope of the appended claims, including the equivalents thereof available as a matter of law.

The patentees do not intend to dedicate any disclosed embodiments to the public, and to the extent any disclosed modifications or alterations may not literally fall within the scope of the claims, they are considered to be part hereof under the doctrine of equivalents.

It is to be understood that each component, compound, substituent or parameter disclosed herein is to be interpreted as being disclosed for use alone or in combination with one or more of each and every other component, compound, substituent or parameter disclosed herein.

It is also to be understood that each amount/value or range of amounts/values for each component, compound, substituent or parameter disclosed herein is to be interpreted as also being disclosed in combination with each amount/value or range of amounts/values disclosed for any other component(s), compounds(s), substituent(s) or parameter(s) disclosed herein and that any combination of amounts/values or ranges of amounts/values for two or more component(s), compounds(s), substituent(s) or parameters disclosed herein are thus also disclosed in combination with each other for the purposes of this description.

It is further understood that each range disclosed herein is to be interpreted as a disclosure of each specific value within the disclosed range that has the same number of significant digits. Thus, a range of from 1-4 is to be interpreted as an express disclosure of the values 1, 2, 3 and 4.

It is further understood that each lower limit of each range disclosed herein is to be interpreted as disclosed in combination with each upper limit of each range and each specific value within each range disclosed herein for the same component, compounds, substituent or parameter. Thus, this disclosure to be interpreted as a disclosure of all ranges derived by combining each lower limit of each range with each upper limit of each range or with each specific value within each range, or by combining each upper limit of each range with each specific value within each range.

Furthermore, specific amounts/values of a component, compound, substituent or parameter disclosed in the description or an example is to be interpreted as a disclosure of either a lower or an upper limit of a range and thus can be combined with any other lower or upper limit of a range or specific amount/value for the same component, compound, substituent or parameter disclosed elsewhere in the application to form a range for that component, compound, substituent or parameter.

1. A composition for oral application comprising silver diamine fluoride and a thickening agent, wherein the thickening agent is in an amount of 0.1 to 15 wt. %, based on a total weight of the composition, wherein said composition is thixotropic and has a viscosity of 0.02 to 500 Pa·s, as measured by a TA AR-G2 rheometer with a 40 mm cone and plate, carried out at a temperature of about 23° C., and a shear rate of from 0.1/second to 100/second.

2. (canceled)

3. The silver diamine fluoride composition of claim 1, wherein the silver diamine fluoride is in a concentration of from 10 wt. % to 40 wt. %, based on a total weight of the composition.

4-8. (canceled)

9. The silver diamine fluoride composition of claim 51, wherein the thickening agent is a cellulose derivative (“cellulose gum”); polyvinyl pyrrolidone; xanthan; an admixture of glycerin and polyacrylate; carrageenans; guar gum; gum karaya; gum arabic; gum tragacanth; silica thickeners and mixtures thereof.

10. (canceled)

11. The silver diamine fluoride composition of claim 51, wherein the thickening agent is selected from carboxy methyl cellulose (CMC), microcrystalline cellulose, and an admixture of glycerin and polyacrylate.

12. (canceled)

13. The silver diamine fluoride composition of claim 1, prepared by mixing silver diamine fluoride with the thickening agent, wherein the silver diamine fluoride is in an amount of about 38 wt. % and the thickening agent is carboxy methyl cellulose (CMC) in an amount of 0.1 to 5 wt. % based on the total weight of the composition.

14. (canceled)

15. The silver diamine fluoride composition of claim 1, further comprising a clathrate of glyceryl acrylate and glyceryl polyacrylate that encloses water molecules, or a cyclodextrin.

16. (canceled)

17. The silver diamine fluoride composition of claim 15, wherein the clathrate is present in an amount of from 0.1 to 15 wt. %, based on the total weight of the composition.

18. (canceled)

19. The silver diamine fluoride composition of claim 1, further comprising a coloring agent.

20. (canceled)

21. The silver diamine fluoride composition of claim 19, wherein the coloring agent is triarylmethane dye.

22. (canceled)

23. The silver diamine fluoride composition of claim 1, further comprising a flavoring agent.

24. (canceled)

25. The silver diamine fluoride composition of claim 1, wherein the composition is a thixotropic fluid that can undergo an isothermal gel-sol-gel transformation.

26. (canceled)

27. The silver diamine fluoride composition of claim 1, wherein the composition has a pH of from 7.0 to about 13.5.

28. (canceled)

29. The silver diamine fluoride composition of claim 1, wherein the viscosity is greater than 75 Pa·s at a shear rate of 0.1/sec and less than 15 Pa·s at a shear rate of 100/sec.

30. (canceled)

31. The silver diamine fluoride composition of claim 1, wherein the silver diamine fluoride provides 10,000 ppm to 100,000 ppm fluoride to the silver diamine fluoride composition.

32. (canceled)

33. The silver diamine fluoride composition of claim 1, wherein the silver diamine fluoride provides 50,000 ppm fluoride to the silver diamine fluoride composition.

34. (canceled)

35. A method of treating teeth, comprising applying the composition claim 1 to a decayed portion of tooth.

36. (canceled)

37. A method of preventing caries in high-risk teeth, comprising applying an effective amount of the composition claim 1 to a surface of the tooth at a high risk of forming a carious lesion.

38. (canceled)
